(19) United States
(12) Patent Application Publication
(10) Pub. No.: US 2016/0128779 A1
(43) Pub. Date: May 12, 2016

(54) WASHCLOTH FOR MEATAL AREA CLEANING OF AN INDWELLING URINARY CATHETERIZED PATIENT

(71) Applicant: Sage Products, LLC, Cary, IL (US)

(72) Inventors: Jennifer M. Allen, Village of Lakewood, IL (US); Gregory T. Davis, Woodstock, IL (US); Jessica A. Farrell, Overveen (NL); Wendee E. Green, Cary, IL (US); Curtis L. Hollabaugh, Huntley, IL (US)

(73) Assignee: Sage Products, LLC, Cary, IL (US)

(21) Appl. No.: 14/881,716

(22) Filed: Oct. 13, 2015

Related U.S. Application Data
(60) Provisional application No. 62/072,155, filed on Oct. 29, 2014.

(51) Int. Cl. A47K 7/03; B65D 85/00; B65D 75/58; B65D 75/56

(52) U.S. Cl. CPC A61B 19/0264; B65D 75/5805; B65D 75/566; B65D 85/70; A47K 7/03; A61M 25/0017

(57) ABSTRACT

A cleaning product for an indwelling urinary catheterized patient including an outer packaging film and at least one washcloth disposed within the film. The at least one washcloth is infused with a cleaning solution that is compatible with the indwelling urinary catheter. A method of using the cleaning product for an indwelling urinary catheterized patient including providing an outer packaging film and at least one washcloth disposed within the film that is infused with a cleaning solution that is compatible with an indwelling urinary catheter, opening the outer packaging film to expose the at least one washcloth, removing the at least one washcloth from the outer packaging film, and cleaning a meatal and surrounding area of the patient with the at least one washcloth.

### RELATED APPLICATIONS

The present patent document claims the benefit of the filing under 35 U.S.C. §119(e) of Provisional U.S. Patent Application Ser. No. 62/072,155, filed Oct. 29, 2014, which is hereby incorporated by reference.

### TECHNICAL FIELD

The present invention relates to a cleaning product for the skin in contact with and surrounding a medical device, the cleaning product having a sealed outer package and at least one disposable washcloth disposed within the package. The washcloth is infused with a cleaning solution being generally uniformly distributed throughout the washcloth and is formed of a cloth comprising cellulose and polyester fibers. The washcloth and packaging are designed such that they facilitate using a separate and clean procedure to clean the skin in contact with and surrounding a medical device to help reduce cross-contamination that could cause device-related infection.

### BACKGROUND

In a healthcare setting, patients may be fitted with an indwelling urinary catheter to help facilitate control and measurement of urine output. One type of indwelling catheter, most often referred to as a Foley catheter, is designed to remain in place for up to 30 days. The Foley catheter is inserted into the urethra through the urinary meatus. It extends into the bladder where a balloon at the distal end of the catheter is inflated to help it remain in place. Any patient with a Foley catheter in place for more than a few days is at high risk of developing catheter-associated urinary tract infections (CAUTI).

CAUTI are one of the most common healthcare-associated infections. The source of bacteria causing CAUTI can be endogenous (typically meatal, rectal, or vaginal colonization) or exogenous (typically the contaminated hands of healthcare personnel, equipment, or materials). The incidence of CAUTI may be reduced with recommended infection control measures addressing both the endogenous and exogenous bacterial sources.

Regardless of the source, the catheter facilitates entry of the bacteria into the urinary tract in two ways: intraluminally or extraluminally. Infraluminal contamination occurs when the internal surfaces of the urinary drainage system become contaminated, allowing the bacteria to enter the urinary tract through the inner lumen of the catheter. Extraluminal contamination occurs when bacteria enter the urinary tract through the urinary meatus via the outer surface of the catheter and/or the urethral tissue. Extraluminal contamination is the mechanism affected by the product and methods described herein.

Cleaning of the meatal area is an important practice to address the bacteria that may cause extraluminal contamination. Clinical Practice Guidelines such as the Centers for Disease Control and Prevention's Guideline for Prevention of Catheter-Associated Urinary Tract Infections 2009 and The Joint Commission's Clinical Care Improvement Strategies: Preventing Catheter-Associated Urinary Tract Infections recommend cleaning the meatal area at least once daily and after a bowel movement. To avoid introducing bacteria during this cleaning, cleaning of the meatal and surrounding area should be a “clean” procedure that is separate from other cleaning routines (e.g. daily bath or incontinence cleanup). However, this often does not occur in current practice. When using a basin bath to clean the patient's body, the healthcare professional often uses the same washcloth, water, and basin that he or she used for the bath to wipe the meatal area without changing his or her medical gloves or performing hand hygiene before cleaning the meatal area. Similarly, when a prepackaged bathing system is used to clean the patient's body, the healthcare professional often uses the same cloth or a new fresh washcloth from the same, now potentially contaminated, prepackaged system without changing his or her medical gloves or performing hand hygiene before cleaning the meatal area. Even the use of a new, fresh washcloth from the same prepackaged system without the healthcare professional changing his or her medical gloves or performing hand hygiene may contaminate the new washcloth before it is used to clean the meatal area because both the package and hands may be contaminated with bacteria. These current procedures may introduce bacteria to the meatal area and increase the risk of CAUTI.

### BRIEF SUMMARY

To decrease the risks of CAUTI, after completing other cleaning routines, a healthcare professional should remove his or her medical gloves, perform hand hygiene, put on a new pair of clean medical gloves, and then clean the meatal area with a fresh, clean product.

Embodiments may include a cleaning product for the urinary meatus and surrounding area that includes a sealed outer package and at least one washcloth disposed within the package. The at least one washcloth is infused with a solution that cleans the skin directly at the meatal-catheter junction and the area immediately surrounding.

In further embodiments, the solution of the at least one washcloth is compatible with an indwelling urinary catheter. Compatibility with the catheter is determined based on testing the catheter after contact with the solution to identify whether the solution causes any erosion, discoloration, damage, or otherwise affects the performance of the catheter.

Another embodiment may include a method of using the cleaning product for routine meatal cleaning. After cleaning a patient's body using a prepackaged bathing product or bath basin, the healthcare professional removes his or her medical gloves, performs hand hygiene, and puts on a non-used pair of medical gloves to reduce the transfer of bacteria from the patient's body or environment (including the bathing materials previously used) to the meatus and surrounding area and subsequently to the indwelling urinary catheter. The cleaning product is then provided to clean the meatus and surrounding area. The outer sealed package of the cleaning product is opened, and one of the at least one washcloths is removed. The meatal and surrounding area is then cleaned using one of the at least one washcloths. The washcloth is then discarded to reduce the potential for cross-contamination from re-use.

### BRIEF DESCRIPTION OF THE DRAWINGS

FIG. 1 shows a front view of the cleaning product.

FIG. 2 shows a back view of the cleaning product.

FIG. 3 shows a cross sectional side view of the cleaning product.

FIG. 4 shows a side view of one of the at least one washcloths of the cleaning product.

FIG. 5 shows a front view of the product dispenser including at least one cleaning product.

FIG. 6 shows a front view of the product dispenser coupled with a retaining component.

### DETAILED DESCRIPTION

Embodiments are described with reference to the drawings in which like elements are generally referred to by like numerals. The relationship and functioning of the various elements of the embodiments may better be understood by referenced to the following description. However, embodiments are not limited to those illustrated in the drawings. It should be understood that the drawings are not necessarily to scale, and in certain circumstances details may have been omitted that are not necessary for an understanding of embodiments of the present invention, such as—for example—conventional fabrication and assembly.

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

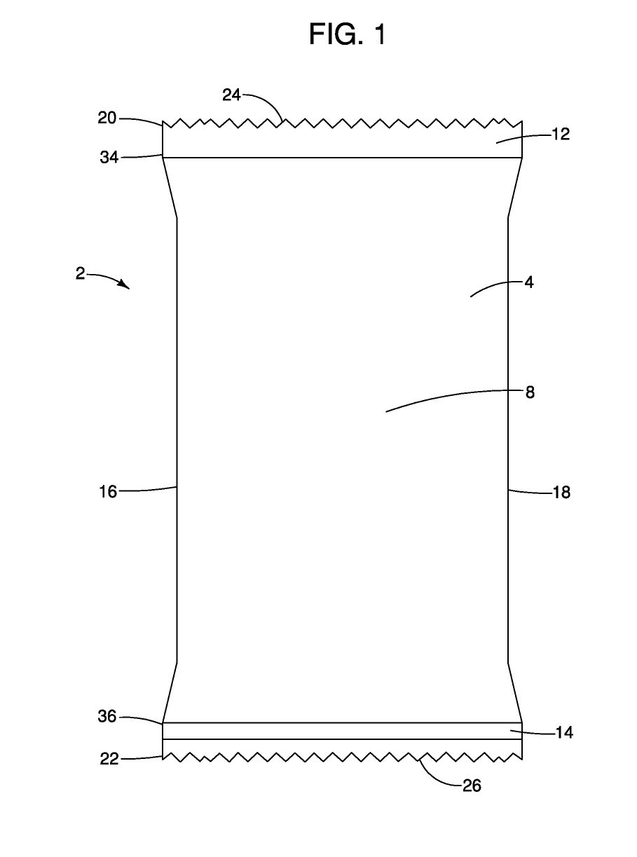

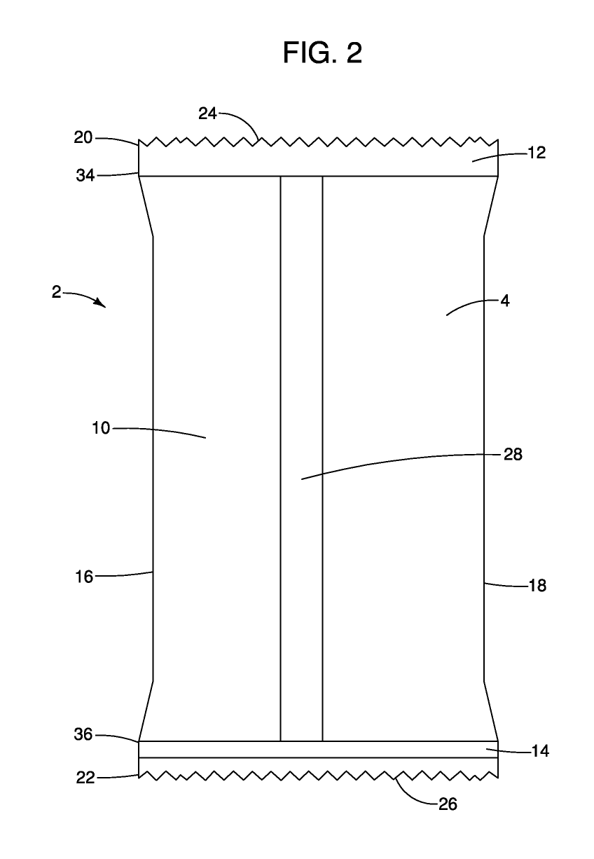

FIGS. 1-4 depict an embodiment of a cleaning product 2 that includes an outer packaging film 4 and at least one washcloth 6 disposed within the film 4. The outer packaging film 4 includes a front side 8, a back side 10, a top portion 12, a bottom portion 14, a left side 16, and a right side 18. The outer packaging film 4 may comprise a polymeric material. The top portion 12 and the bottom portion 14 are sealed and include sealed edges 34, 36. The top portion 12 and the bottom portion 14 are sealed via a heat sealing process. The top portion 12 includes an edge 20, and the bottom portion 14 also includes an edge 22. The edges 20, 22 include a plurality of end cuts 24, 26. The plurality of end cuts 24, 26 enable tearing open the outer packaging film 4 to expose the at least one washcloth 6. As illustrated in FIG. 2, the outer packaging film 4 also includes a fin seal 28 that extends along the back side 10 of the outer packaging film 4. The fin seal 28 includes a tear notch 30 that enables tearing open the fin seal to expose the at least one washcloth 6. The tear notch 30 may be located approximately 1.5 to 2.5 inches from the edge 20 of the top portion 12 of the outer packaging film 4. The outer packaging film 4 is preferably not self-resealable to encourage or remind healthcare workers to dispose of the cleaning product 2 after each use rather than save remaining washcloths 6 for use in a future cleaning session. The term “self-resealable” includes adhesive flaps that are easily resealable by closing the flap against the packaging. The term “self-resealable” also includes any other features that make it easy for a user to reseal the packaging without using external components that is not provided with the packaging, such as a piece of tape.

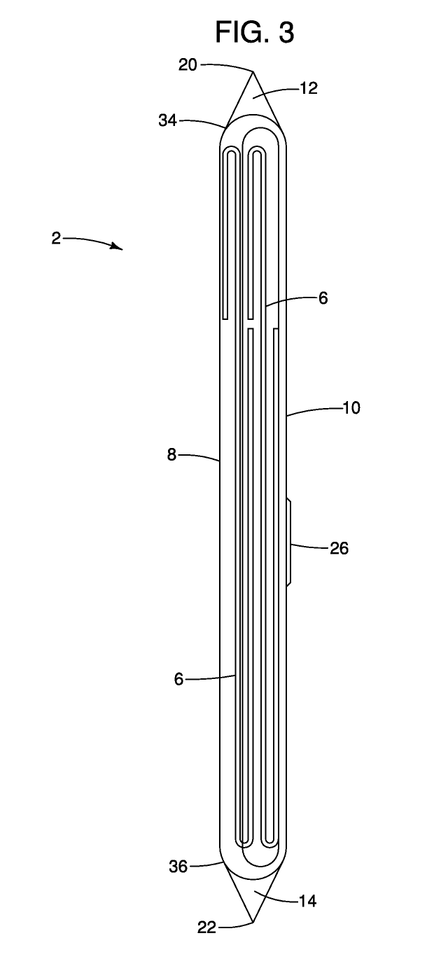

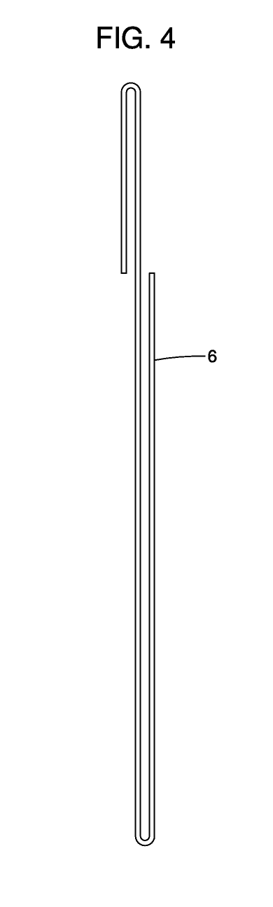

The at least one washcloth 6 illustrated in FIGS. 3-4 may include a Z-shape formation when disposed within the outer packaging film 4. After removal from the outer packaging film 4, the at least one washcloth 6 may be unfolded and include a rectangular shape. The at least one washcloth 6 is preferably non-woven. The at least one washcloth 6 preferably includes cellulose and polyester mixed fibers. The at least one washcloth 6 may include approximately 41% to 59% by weight cellulose fiber and the remaining percentage may include polyester fibers, and may be a 50% cellulose and 50% polyester mixed fiber. The thickness of the at least one washcloth 6 may range from 0.040 to 0.080 inches, and in one embodiment is approximately 0.057 inches. The weight of the at least one washcloth 6 may range from 2.8 to 3.8 ounces per square yard, and is in one embodiment approximately 3.3 ounces per square yard.

The at least one washcloth 6 illustrated in FIGS. 3-4 is infused with a solution 32 for cleaning the meatal area of a catheterized patient. The pH of the solution 32 may range from 4.5 to 6.0.

In addition to being a clean and separate procedure, the at least one washcloth 6 infused with the solution 32 should: 1) be safe for use on keratinized and partially keratinized skin as well as mucous membranes; 2) release sufficient solution to clean the meatal and surrounding area such that excess solution is minimized to avoid the meatal and surrounding area being wet after cleaning, as this can promote bacterial growth; and 3) be compatible with the urinary catheter such that the at least one washcloth 6 infused with the solution 32 does not erode, discolor, damage, or otherwise impact the performance of the catheter. Compatibility with keratinized and partially keratinized skin includes lack of or minimal irritation with the skin or any adverse reaction to the skin after exposure to the at least one washcloth 6 infused with the solution 32.

The at least one washcloth 6 infused with the solution 32 is compatible with a Foley catheter. Compatibility with a Foley catheter includes testing that may include without limitation visual surface inspections, measuring changes in the outer diameter of the shaft, the flow rate of the catheter, deflation reliability of the balloon, and the tensile strength of the catheter. For example, The American Society for Testing and Materials' (“ASTM International”) Standard Performance Specification for Foley Catheter, F623-99 (Reapproved 2013) identifies various standards and tests for a Foley catheter to meet industry standards including the flow rate of the Foley catheter and deflation reliability of the balloon in the Foley catheter. For testing flow rates, testing may be run for 14 or 30 days, and the Foley catheter is treated with the at least one washcloth 6 infused with the solution 32 at least four times per day. The flow rate of the Foley catheter may be measured each day prior to the first treatment, immediately following the final treatment, and one hour after the final treatment. Flow rates of the Foley catheter after treatment of the at least one washcloth 6 infused with the solution 32 exceed 300 cm³/min. Testing of deflation reliability of the balloon in the Foley catheter may be used to determine the reliability of the balloon deflation after simulated use for one week. Testing may be conducted for 14 or 30 days, and balloon deflation and burst percentage of the Foley catheter after treatment of the at least one washcloth 6 infused with the solution 32 has been the same or better than as the untreated catheter.

Testing relating to visual surface inspections, measuring changes in the outer diameter of the shaft, and the tensile strength of the Foley catheter may also be used to identify compatibility. For visual surface inspection, testing may be run for 30 days, and the Foley catheter is treated with the at least one washcloth 6 infused with the solution 32 at least four times per day. Visual inspections, including changes in catheter color, surface damage, or other visually identifiable changes to the surface of the catheter under low magnification, are taken after each treatment each day. A Foley catheter exposed to the at least one washcloth 6 infused with the solution 32 according to this treatment shows no visible changes in 95% of visual observations.

Testing may also be conducted for measuring changes in the outer diameter of the shaft of the Foley catheter, as swelling or increasing the size of the outer diameter of the shaft may cause urethral damage. The outer diameter testing may be conducted for 30 days, and the Foley catheter is treated with the at least one washcloth 6 infused with the solution 32 at least four times per day. Measurements of the outer diameter of the shaft are taken after each treatment each day. An outer diameter of the shaft of a Foley catheter exposed to the at least one washcloth 6 infused with the solution 32 according to this treatment may range from 0 to 2 French size greater than the untreated catheter, which is the same as the current cleansing treatment with soap and water.

Testing may also be conducted for measuring the tensile strength of the Foley catheter after treatment with the at least one washcloth 6 infused with the solution 32. In this testing, the Foley catheter is treated 4 times per day, where a single treatment includes wiping the catheter several times with either soap and water or the at least one washcloth 6. The catheters may be permitted to air dry for at least 15 minutes between treatments. The catheter is then put under tension until the point at which it breaks or reaches maximum elongation. The test is performed after 14 days of treatment and after 30 days of treatment. The tensile strength of a Foley catheter exposed to the at least one washcloth 6 infused with the solution 32 has been the same as a Foley catheter treated with normal soap and water.

A method of using the cleaning product 2 may include after cleaning a patient's body using washcloths infused with a cleansing solution, the healthcare professional removes his or her medical gloves, performs hand hygiene, and puts on a non-used pair of medical gloves to reduce the transfer of bacteria from the patient's body to the meatal and surrounding area and subsequently to the catheter. The cleaning product 2 is then provided to clean the meatal and surrounding area. The outer packaging film 4 of the cleaning product 2 is opened, and one of the at least one washcloths 6 is removed. The meatal and surrounding area is then cleaned using one of the at least one washcloths 6 and subsequently discarded to reduce the potential for cross-contamination from re-use. The small number of washcloths 6 in each outer packaging film 4 will help encourage or remind health care workers to discard the cleaning product 2 after each use, thereby causing the healthcare worker to open a fresh package of cleaning product 2 before each use. This step may also encourage or remind the healthcare worker to use a fresh, unused pair of gloves before taking the step of opening a fresh package of cleaning product 2.

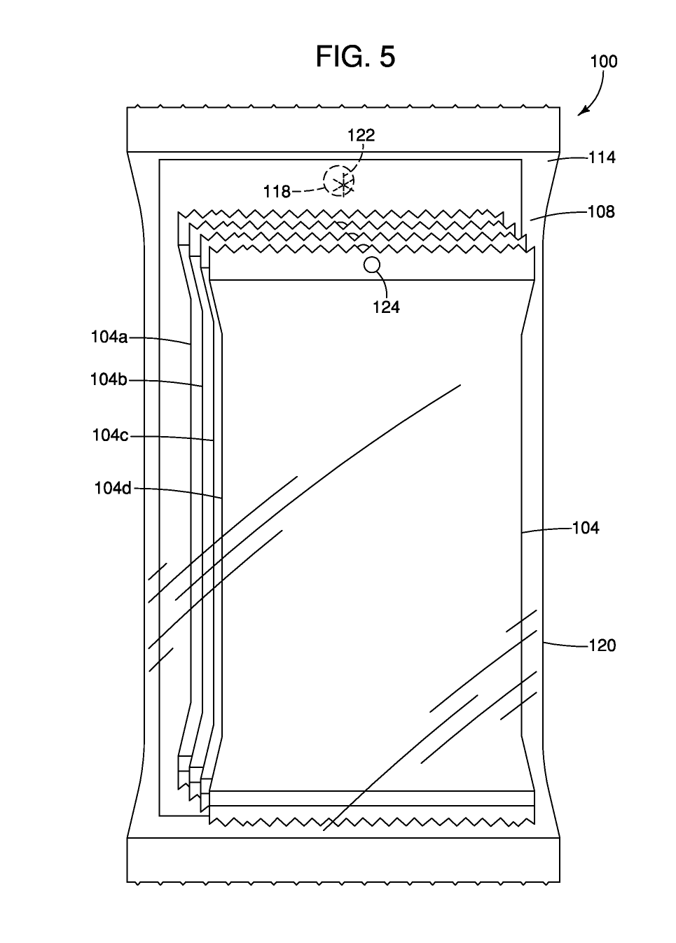

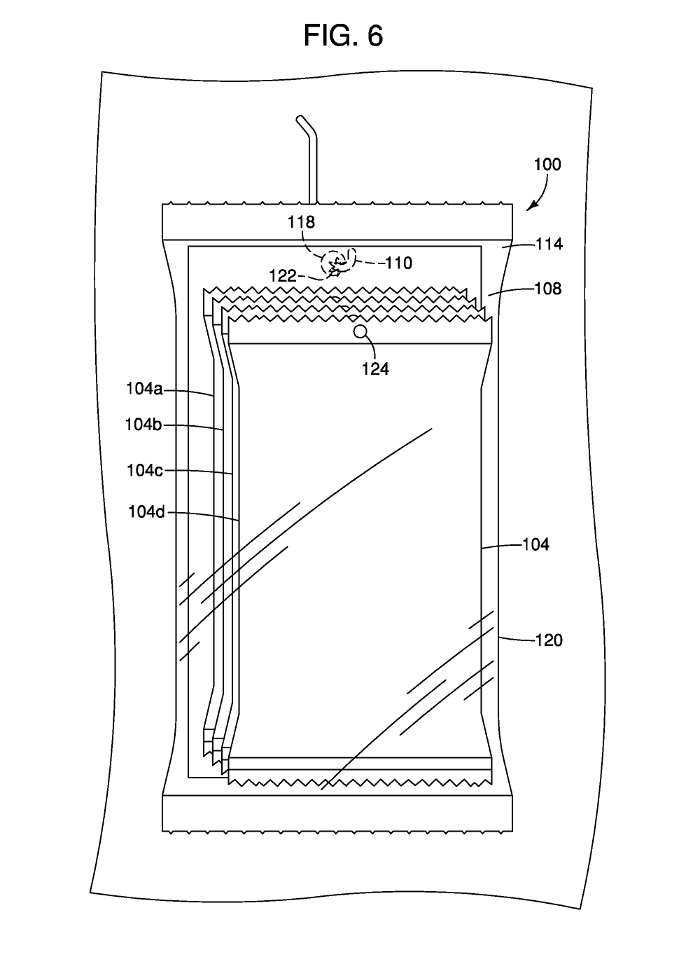

As shown, for example, in FIGS. 5-6, the cleaning product 2 may also include a cleaning product dispenser 100 having a hanging element 108 and at least one outer packaging film 104 disposed within the hanging element 108. As with prior embodiments, the outer packaging film 104 includes at least one washcloth disposed within (not shown in FIGS. 5-6). The hanging element 108 may be used for hanging the cleaning product dispenser 100 from a location proximal to the patient and/or healthcare worker. For example, it may be desirable to hang the cleaning product dispenser 100 on a wall next to the patient's bed or from the patient's bed itself. It may also be desirable to hang the cleaning product dispenser 100 near the healthcare worker's other cleaning supplies, such as disposable gloves.

The cleaning product dispenser 100 includes at least one outer packaging film 104 and preferably several. For example, the cleaning product dispenser 100 shown in the figures includes four packs 104a, 104b, 104c, 104d of outer packaging film 104. This number of packs is particularly desirable because the catheter is treated with the at least one washcloth at least four times per day. The cleaning product dispenser 100 may have a different number of packs, particularly if the number of packs is not so great that it makes it impractical to hang the cleaning product dispenser 100. Each outer packaging film 104a, 104b, 104c, 104d may also include a hole 124 to allow each outer packaging film 104a, 104b, 104c, 104d to be hung on its own or otherwise independent from the hanging element 108.

The hanging element 108 is able to selectively mate with a hook 110 (FIG. 6). The hanging element 108 may also be used with a clip or other suitable fastener. The hanging element 108 shown in the figures includes a bag 114 with a thru-hole 118 for receiving a hook 110. The thru-hole 118 is located on the back side of the bag. The hanging element 108 may also be secured with a clip or other appropriate fastener. The hanging element shown in FIGS. 5-6 also includes a packaging insert 120 to stiffen the packaging and support the weight of the cleaning product dispenser 100 when hanging. The packaging insert 120 shown in the figures is made of cardboard, but any lightweight material may be used, such as plastic. The packaging insert 120 includes a plurality of retainer flaps 122 that permit the hook 110 or clip to penetrate the packaging insert 120 while also holding the cleaning product dispenser 100 onto the hook 110 or clip. For example, the retainer flaps 122 shown in FIGS. 5-6 are formed via a plurality of cuts in the packaging insert 120 so as to form a star-shaped plurality of flaps. Thus, when the cleaning product dispenser 100 is hanging from the hook 110, the hook 110 penetrates through the thru-hole 118 of the bag 114 and the retainer flaps 122 of the packaging insert 120, which allows the clinician to access the outer packaging films 104a, 104b, 104c, 104d when the cleaning product dispenser 100 is hanging.

As shown in FIG. 6, a top portion of the bag 114 was removed before hanging the product dispenser 100 on the hook 110 so that a healthcare worker may easily gain access to the individual packaging films 104. The bag 114 may include perforations to facilitate such removal. Alternatively, only a portion of the bag 114 may include perforations such that the top portion of the bag 114 may be folded back rather than completely removed.

Those skilled in the art will appreciate that embodiments not expressly illustrated herein may be practiced within the scope of the present invention, including that the features described herein for different embodiments may be combined with each other and/or with currently-known or future-developed technologies while remaining within the scope of the claims presented herein. Although specific terms are employed herein, they are used in a generic and descriptive sense only and not for purposes of limitation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment of the invention.

1. A cleaning product for an indwelling urinary catheterized patient comprising:
an outer packaging film and at least one washcloth disposed within the film, wherein the at least one washcloth is infused with a cleaning solution that is compatible with an indwelling urinary catheter.

2. A cleaning product as recited in claim 1, wherein the outer packaging film is not self-resealable.

3. A cleaning product as recited in claim 2, wherein the outer packaging film includes at least one end configured to enable tearing open the outer packaging film.

4. A cleaning product as recited in claim 2, wherein the outer packaging film includes at least one fin seal configured to enable tearing open the outer packaging film.

5. A cleaning product as recited in claim 4, wherein the at least one fin seal extends along the outer packaging film.

6. A cleaning product as recited in claim 5, wherein the at least one fin seal includes a tear notch.

7. A cleaning product as recited in claim 1, wherein the at least one washcloth consists of two washcloths.

8. A cleaning product as recited in claim 1, wherein the at least one washcloth consists of less than five washcloths.

9. A cleaning product as recited in claim 1, wherein the at least one washcloth consists of less than ten washcloths.

10. A cleaning product dispenser for an indwelling urinary catheterized patient comprising:
an outer packaging film;
at least one washcloth disposed within the outer packaging film; and
a hanging element coupled with the outer packaging film and configured to enable hanging the outer packaging film.

11. A cleaning product dispenser as recited in claim 10, wherein the hanging element includes a bag.

12. A cleaning product dispenser as recited in claim 11, wherein the bag includes at least one thru-hole.

13. A cleaning product dispenser as recited in claim 10, wherein the hanging element includes a packaging insert.

14. A cleaning product dispenser as recited in claim 13, wherein the packaging insert is cardboard.

15. A cleaning product dispenser as recited in claim 13, wherein the packaging insert includes at least one thru-hole.

16. A cleaning product dispenser as recited in claim 13, wherein the packaging insert includes retainer flaps.

17. A cleaning product dispenser as recited in claim 10, wherein the hanging element includes at least one thru-hole.

18. A method of using a cleaning product for an indwelling urinary catheterized patient comprising:
providing an outer packaging film and at least one washcloth disposed within the outer packaging film, wherein the at least one washcloth is infused with a cleaning solution that is compatible with a Foley catheter;
opening the outer packaging film to expose the at least one washcloth;
removing the at least one washcloth from the outer packaging film; and
cleaning a meatal and surrounding area of the patient with the at least one washcloth.

19. A method of using a cleaning product as recited in claim 18, further comprising discarding the outer packaging film and the at least one washcloth after a single use.

20. A method of using a cleaning product as recited in claim 18, further comprising hanging the outer packaging film proximal to the patient.
